(19) United States
(12) Patent Application Publication
(10) Pub. No.: US 2019/0043606 A1
(43) Pub. Date: Feb. 7, 2019

(54) PATIENT-PROVIDER HEALTHCARE RECOMMENDER SYSTEM

(71) Applicant: Teladoc, Inc., Purchase, NY (US)

(72) Inventors: Kurt Roots, Madison, WI (US); Jeffrey Nadler, Rye Brook, NY (US)

(21) Appl. No.: 16/051,616

(22) Filed: Aug. 1, 2018

Related U.S. Application Data
(60) Provisional application No. 62/541,265, filed on Aug. 4, 2017.

(51) Int. Cl. G16H 10/60; G06N 99/00

(52) U.S. Cl. CPC G16H 10/60; G06N 99/005

(57) ABSTRACT

Systems and methods for providing an optimal healthcare provider match to a patient by a predictive engine employing machine learning.

### FIELD OF THE INVENTION

The present invention relates to a recommender system (RS) for healthcare, also known as a health recommender system (HRS). The HRS of the present invention comprises an advanced architecture and employs concepts from content-based filtering to recommend optimal patient-provider matches utilizing attributes from diverse dimensions such as patients, providers, environment, location, age, gender, and the like. Instead of strictly embodying utility matrices and evaluating prior rankings, the predictive engine of the present invention relies on a variety of descriptive features and machine learning to suggest optimal healthcare providers to the health system patient.

### BACKGROUND OF THE INVENTION

Historically, the decision of selecting a medical provider was found to not be consumeristic or rational, instead relying on family, friends, and other factors based on geographic and historical contexts as the drivers for a recommendation. Harris, How do Patients Choose Physicians? Evidence from a National Survey of Enrollees in Employment-Related Health Plans, Health Services Research, 38(2):711-732 (2017). Studies in the 1990s and early 2000s focused on factors like location, race, gender, and specialty. Saha et al., Do Patients Choose Physicians of their Own Race?, Health Affairs, 19(4):76-83 (2000); Phillips et al., Women Patients' Preferences for Female or Male GPs, Family Practice, 5(6):543-547 (1998). This changed in recent years where a high percentage of patients are aware of and review online provider ratings to make decisions. While these existing intrinsic value decisions are still being applied, a new spectrum of preferences can be used to select a provider. The economics of selection change when new criteria like appearance are applied to the selection process. Puhl et al., The Effect of Physicians' Body Weight on Patient Attitudes: Implications for Physician Selection, Trust and Adherence to Medical Advice, International Journal of Obesity, 37:1415-1421 (2013). Further, this consumerization impacts a range of provider types, including, but not limited to doctors, therapists, dentists, optometrists, chiropractors, and nurses.

This new and more advanced selection process is most frequently driven through consumer facing apps and websites like Yelp (yelp.com) and Healthgrades (healthgrades.com) where users can search and rank experiences with providers. This approach fundamentally relies on other users' rankings, both captured through surveys and written reviews. These services then utilize this information with generalized filtering instruments to provide a listing of providers to the user. Ornstein, On Yelp, Doctors Get Reviewed Like Restaurants—And it Rankles, available at https://www.npr.org/sections/health-shots/2015/08/06/429624187/on-yelp-doctors-get-reviewed-like-restaurants-and-it-rankles (2015).

It is an intuitive, yet novel, progression from non-consumeristic, consumeristic, to sophisticated retail-like personalization of the decision process, wherein providers are accurately presented through advanced artificially intelligent agents to the patient for observation, review, and ultimately selection to deliver care.

Recommender systems have been widely used in industries like retail to provide personalized suggestions to individuals. Typical recommender systems rely on historical data patterns from the consumer to predict future behavior. For example, those streaming movie X might also like movies Y and Z, which are frequently viewed together by others. Digital form factors provide a seamless experience to represent these suggestions to the consumer for evaluation, selection, and purchase.

Recommender systems are traditionally classified as content-based or collaborative filtering approaches. Collaborative filtering is generally concerned with the relationship between users and/or items, wherein the similarity of the items is represented by users typically rating more than one item. Within collaborative filtering, there are two commonly used approaches: item-item and user-based. The former recommends similar items based on target user's prior ratings of similar items, typically while browsing other items. The latter recommends similar items to the target user based on similarities between users and their prior behavior. In summary, collaborative filtering relies on past preferences or rating correlation between users, without reliance on additional user or item data (i.e. metadata). Collaborative filtering suffers from the “cold start” problem and can be problematic in a healthcare setting because it raises the concern of sharing patient rankings and preferences across populations.

Both of the approaches of collaborative filtering differ from content-based filtering, which generally recommends similar items using attributes from profiles. The items in this approach require profiles of attributes to be generated. For example, attributes or metadata of a t-shirt for which a user expressed interest would store things like size, color, brand, and so forth. The system then matches attributes of that t-shirt to other t-shirts viewed with similar attributes to generate recommendations. This method has two primary advantages in a healthcare setting: (1) the isolation of ratings to an individual user's profile; and (2) new items can be easily incorporated (no cold start problem). The primary disadvantage of content-based filtering in this context is that new user ratings or information about a user has to be collected. Thus, returning to the example, if a user never expressed interest in the t-shirt, we do not have information about their preferences. Hao, A Comparative Study: Classification vs. User-Based Collaborative Filtering for Clinical Prediction, BMC Medical Research Methodology, 16:172 (2016); Leskovec et al., Mining of Massive Datasets, Stanford University (2014).

The present invention seeks to solve the aforementioned drawbacks by providing systems and methods that provide proper safeguards for the sensitive nature of healthcare patient information, and impute preferences of patients without prior data regarding the patients' preferences.

Similar to on-demand ridesharing drivers, medical professionals can be made available nearly instantly through a frictionless delivery model. However, unlike most retail situations or hailing a ride to the airport, healthcare is very personal and highly regulated. Additionally, selecting the “best” provider for one's needs can be complicated and is dependent on a state-by-state basis because of provider licensing requirements. This invention personalizes provider selection by advancing the complex and sensitive process of selecting a provider while controlling for geographic complexity.

### SUMMARY OF THE INVENTION

The present invention relates generally to a Health Recommender System and related methods that employ a sophisticated system architecture and technology stack that may be encapsulated in a container platform, and an artificially intelligent agent, also referred to herein as a predictive engine, that can optimally match patients with healthcare providers. As used herein, the term “patient” may be used interchangeably with the term “member” and the term “provider” may be used interchangeably with the terms “physician,” “care provider,” or “caregiver.”

The system and methods of the present invention are designed to efficiently collect, quantify, and evaluate data across several dimensions important to the provider and patient. In an embodiment, the data components undergo a variety of preprocessing and statistical transformations in preparation for processing by the predictive engine. The preprocessed data is then passed to the predictive engine, which applies a supervised learning technique to evaluate a patient-provider relationship and compute a likelihood of match scenario. The predictive engine thus produces optimal provider recommendations for a new or repeat patient. These results are indexed in a matrix and made available in real-time.

The systems and methods of the present invention have several important advantages. One such advantage is the ability to determine if additional expertise or a referral is required for the patient before the consultation or visit occurs. Under a traditional framework, for example, while a patient may experience sinusitis symptoms, they may also be clinically depressed. The secondary complaint may go unnoticed, de-prioritized and/or not communicated to a mental health professional in favor of resolving the chief complaint. Another advantage of the systems and methods of the present invention is their utility in brick-and-mortar clinics, pharmacies, and nursing homes, wherein a pharmacists, nurse, and/or geriatric care provider is optimally determined based on a particular patient's specific needs. Additionally, the systems and methods of the present invention are particularly well-suited for an on-demand healthcare economy, where everything from virtual clinics to on-demand insurance offerings are becoming commonplace.

One embodiment of the present invention is directed to a healthcare recommender system for matching healthcare providers with patients that employs:
one or more provider records, each provider record corresponding to a provider;
one or more patient records, each patient record corresponding to a patient and containing at least one demographic data value of the patient;
a multidimensional feature space relating to one patient and defining one or more patient-provider combinations, wherein each patient-provider combination is made up of a set of values that includes at least the demographic data value of the patient and at least one of an aggregate internal review score of the provider and an aggregate external review score of the provider;
and a predictive engine capable of machine learning that classifies each patient-provider combination of the feature space into one of two or more classes according to the set of values.

Another embodiment of the present invention is directed to a computer-implemented method for matching healthcare providers with patients employing the steps:
(i) creating a provider record corresponding to a provider;
(ii) creating a patient record corresponding to a patient and populating the patient record with at least one demographic data value of the patient;
(iii) at least one of:
   (a) receiving one or more internal data of the provider from one or more historical patients of the provider, and for each internal data creating an internal review record populated with the internal data and a demographic data value of the historical patient. In a preferred embodiment, the internal data is an internal score, which may be generated from a survey completed by the historical patient; and
   (b) retrieving one or more external data of the provider from one or more third party websites or databases, and for each external data creating an external review record populated with the external data and a demographic data value of the source of the external data. In a preferred embodiment, the external data is a review of the provider authored by a reviewer. In a most preferred embodiment, the external data is a computed numerical sentiment value by natural language processing of the review. In an embodiment, the demographic data value of the reviewer is determined as discussed below where it is not provided;
(iv) generating a multidimensional feature space relating to one patient and defining one or more patient-provider combinations, wherein each patient-provider combination is made up of a set of values that at least includes the demographic data value of the patient and at least one of an aggregate internal review score and an aggregate external review score. In a preferred embodiment, the aggregate internal review score is computed from the internal review records having a demographic data value of the historical patient that is the same as the demographic data value of the patient, and the aggregated external review score is computed from the external review records having a demographic data value of the reviewer that is the same as the demographic data value of the patient; and
(v) classifying by a predictive engine capable of machine learning each patient-provider combination of the feature space into one of two or more classes according to the set of values.

Steps (ii), (iii)(a), and (iii)(b) of the above method may be performed in any order.

A further embodiment of the present invention is directed to a computer-implemented method for matching a patient with one or more healthcare providers employing the steps:
(i) receiving a request from the patient to be matched with the providers, wherein the patient has a corresponding patient record that includes at least one demographic data value;
(ii) generating a multidimensional feature space relating to the patient and defining one or more patient-provider combinations, wherein each patient-provider combination is made up of a set of values that at least includes the demographic data value of the patient and at least one of an aggregate internal review score of the provider and an aggregate external review score of the provider;
(iii) classifying by a predictive engine capable of machine learning each patient-provider combination of the feature space into one of two or more classes according to the set of values. In a preferred embodiment, at least one class represents optimal provider matches;
(iv) presenting to the patient one or more providers, preferably those classified in the class representing optimal provider matches; and
(v) receiving a selection of one provider from the patient.

The providers may be presented to the patient in a graphical user interface (GUI) that preferably includes a digital image of the provider and biographical information about the provider such as name, age, gender, geographical location, and medical specialty. After the patient's selection of the provider is received, the methods of the present invention may further include the step of enabling communication between the patient and the provider via a network, wherein the communication may be text, audio, video, or a combination thereof.

In embodiments of the present invention, the aggregate internal review score of the provider is derived from one or more internal review records corresponding to the provider, each internal review record including an internal score provided by a historical patient of the provider and a demographic data value of the historical patient, and the aggregate internal review score of the provider is computed from the internal review records having a demographic data value of the historical patient that is the same as the demographic data value of the patient. In preferred embodiments of the present invention, the internal review score is derived from structured surveys completed by historical patients of the provider, i.e., patients that have previously had a consultation or other interaction with the provider.

In embodiments of the present invention, the aggregate external review score of the provider is derived from one or more external review records corresponding to the provider, each external review record including an external score from a third party website or database and a demographic data value of the source of the external score, and the aggregate external review score of the provider is computed from the external review records having a demographic data value of the source that is the same as the demographic data value of the patient. In a preferred embodiment the external score is a review of the provider authored by a reviewer. In a most preferred embodiment, the external data is a computed numerical sentiment value of the review by natural language processing of the review. In an embodiment, the demographic data value of the reviewer is determined as discussed below where it is not provided.

### BRIEF DESCRIPTION OF THE DRAWINGS

FIG. 1 is one embodiment of a construct and overview of the provider recommender engine in accordance with the present invention, indicating provider and patient profiles that form a composite feature space for prediction.

FIG. 2 is a diagram of an embodiment of the data workflow for the provider recommender system in accordance with the present invention.

FIG. 3 is a diagram of an embodiment of endpoints showing how the provider recommender system communicates with external services and subsystems in accordance with the present invention.

FIG. 4 is a diagram of an embodiment of the interaction between external application services and the provider recommender system in accordance with the present invention.

FIG. 5 is a diagram of an embodiment of the logical requirements within any application service to interact successfully with the provider recommender system in accordance with the present invention.

FIG. 6 is a diagram of an embodiment of the overall subsystems of the provider recommender system in accordance with the present invention.

FIG. 7 is a diagram showing how each of the subsystems within the provider recommender system functions with each other in accordance with an embodiment of the present invention.

FIG. 8 is a diagram showing the structured survey ingestion, transformation, scoring, and storage in accordance with an embodiment of the present invention.

FIG. 9 is a diagram showing the unstructured review scraping and ingestion, storage, matching, sentiment scoring, and result storage in accordance with an embodiment of the present invention.

FIG. 10 is a diagram showing index transformation and class labeling in accordance with an embodiment of the present invention.

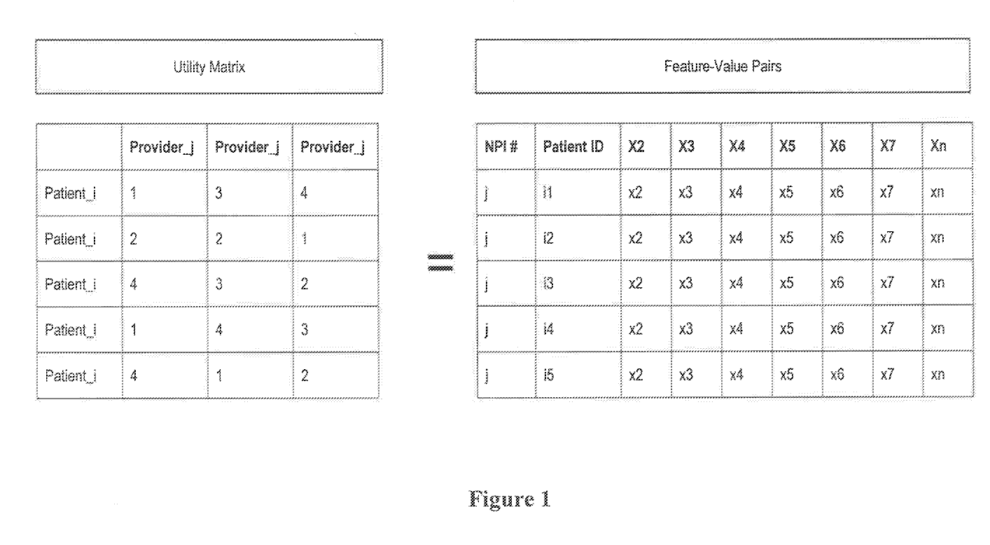

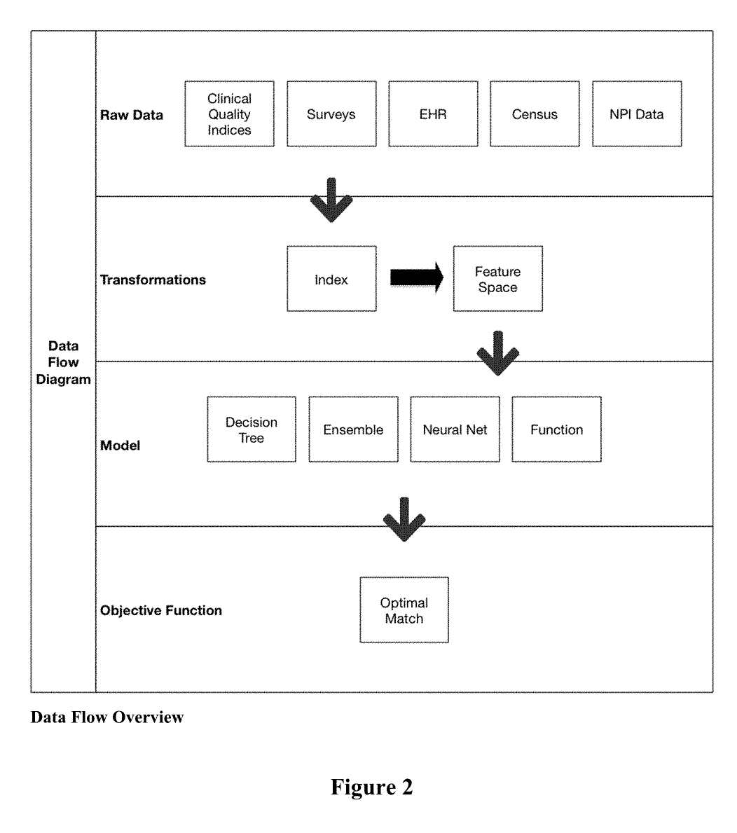

### DETAILED DESCRIPTION OF THE INVENTION

The systems and methods of the present invention are suited for a platform in which patients in need of medical care are connected with healthcare providers. Such a platform may exist as part of a medical practice, hospital, or senior care facility. In a preferred embodiment, the platform is utilized for internet-based medical care where patients and providers register accounts, and registered patients receive on-demand medical care from a registered provider. An example of internet-based medical care is Teladoc (teladoc.com).

In a preferred embodiment, the present invention employs patient records and provider records to keep track of patient and provider data. The patient and provider records are preferably stored in one or more database tables in the non-volatile memory of a computer system. A patient record or a provider record may be composed of data from one or more database tables. Thus, all data in a database, or across multiple databases, relating to a particular patient or a particular provider is considered to form a part of that patient's record or that provider's record, respectively.

Each patient record may contain basic information about a patient such as name, insurance information, and payment information. The patient record according to the present invention also includes one or more demographic data values of the patient, which may be selected from age, age range, city of residence, state of residence, gender, race, ethnicity, medical condition, and the like. The patient record of the present invention may further include medical history, prior provider consultation history, and the patient's prior ratings of providers. In a preferred embodiment, the patient record is populated with at least one demographic data value of the patient, preferably age and/or gender.

Each provider record may contain basic information about the provider such as name, state of medical license, number of years in practice, insurance accepted, and bank information. The provider record may also include one or more demographic data values of the provider, which may be selected from age, age range, city of practice, state of practice, gender, race, ethnicity, medical specialties, and the like. The provider record may further include historical medical outcomes, quality metrics, and prior patient ratings of the provider. In a preferred embodiment of the present invention, the provider record includes a unique government-issued identifier of the provider, which is most preferably the provider's National Provider Identifier (NPI) issued by the Centers for Medicare and Medicaid Services (CMS). An NPI number is a unique 10-digit identification number issued to covered health care providers by the CMS. The NPI database, available at https://npiregistry.cms.hhs.gov, is searchable by fields including NPI number, provider first and last name, and provider organization or medical group name. In an embodiment, the unique government-issued identifier of the provider may serve as a unique index for the provider record. In a preferred embodiment, the provider record is populated with at least the unique government-issued identifier and may also include at least one demographic data value of the provider, which is preferably age and/or gender.

According to the present invention, when a patient requests a healthcare provider, the systems and methods of the present invention seek to provide an optimal match by comparing the patient's record to all relevant physician records and predicting optimal relationships.

The systems and methods of the present invention embody a sophisticated architecture comprising an application programming interface (API) and an artificial intelligence (AI) engine that facilities data ingestion, preprocessing and statistical transformation, feature development and selection, model selection, model training, model execution, and results evaluation. It relies on principles from content-based filtering and classification instead of determining results directly from a utility matrix, as seen in collaborative filtering. This approach does not have the “cold start” problem like collaborative filtering, and further, relies on established attributes of the patient and provider and avoids inspecting user profiles across users, which is a concern for traditional collaborative filtering recommenders in a healthcare context.

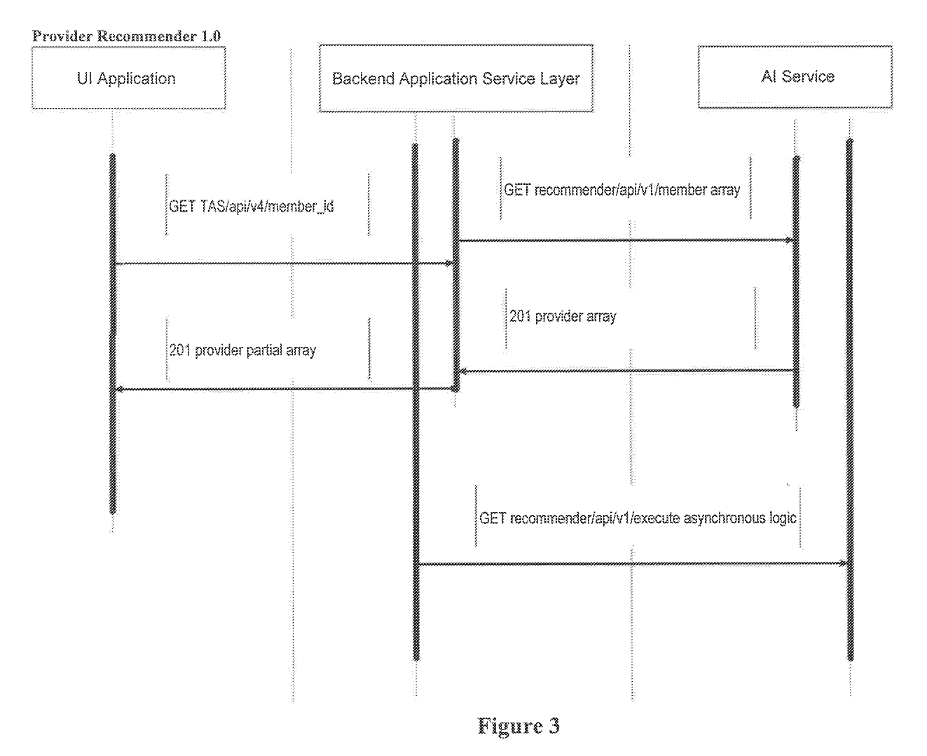

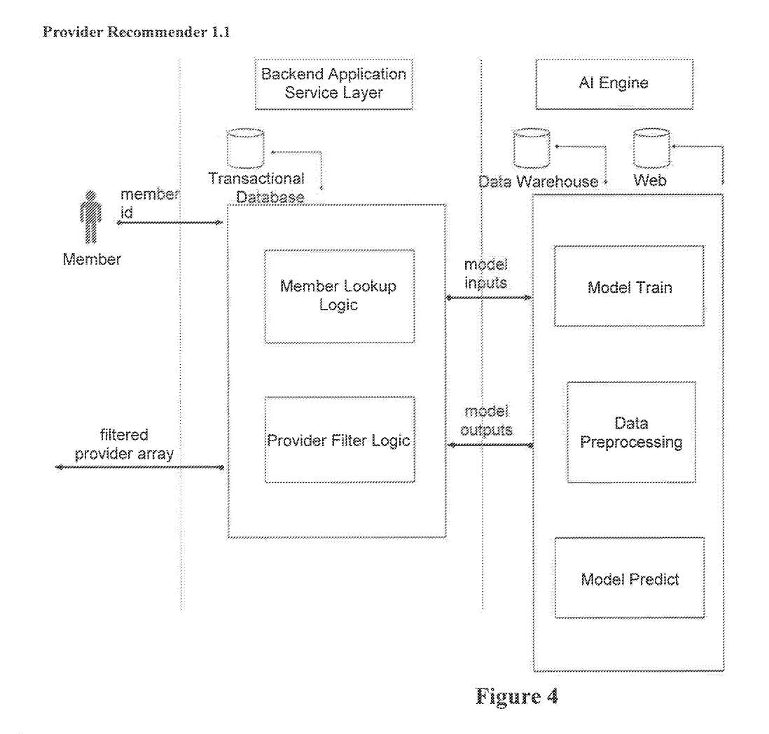

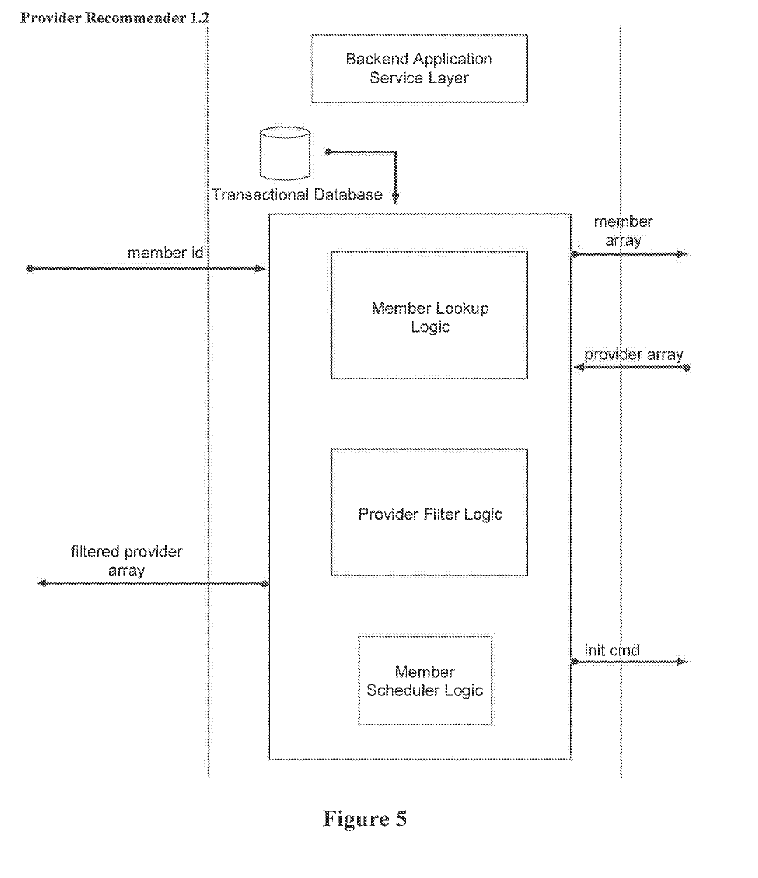

In an embodiment of the present invention, the recommender system may communicate with external cloud microservices through a RESTful API. It encapsulates an interface that accepts standardized web request methods part of the HTTP protocol as the de facto communication mechanism. A variety of external application services, which follow a standard API, can then communicate with the provider recommender system. The provider recommender system has two end-points that enables interaction with the system. The first, allows application services to send initialization commands to the recommender system. As an example, a service such as CRON can asynchronously communicate to the system to train and test the model. The second provides two-way communication of input for model predictions and output of model results. As an example, patient attributes can be sent by the application system and consumed by the provider recommender engine for model prediction and scoring. The predictive results along with statistical measures of relevancy can then be sent back to the application services through the same endpoint as shown in FIG. 3.

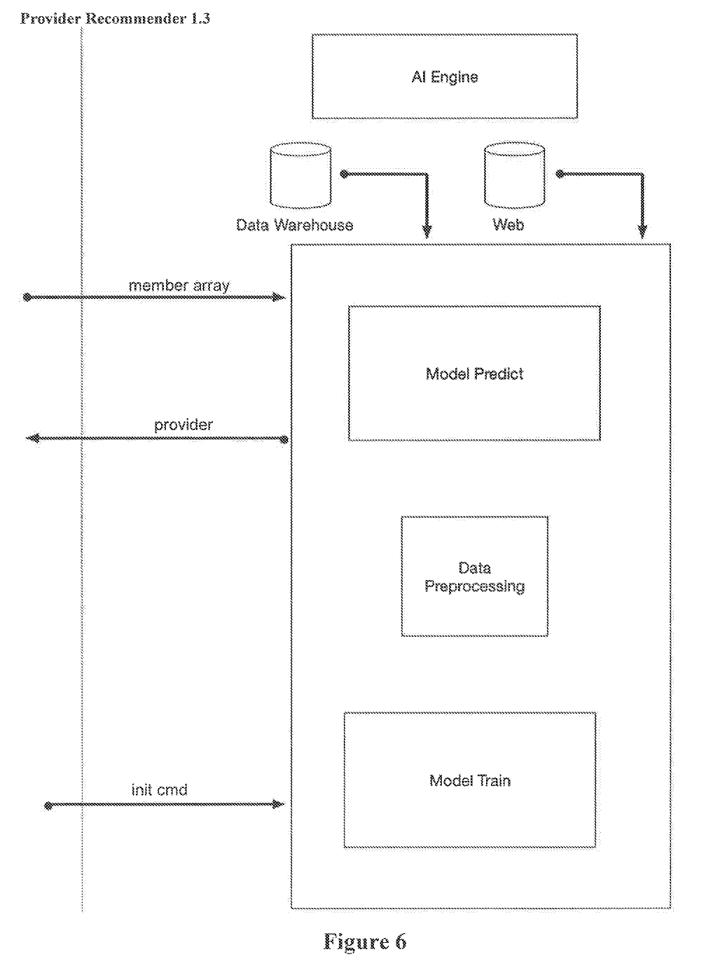

According to the systems and methods of the present invention, the predictive capabilities function through an AI engine, or predictive engine, which consists of three primary subsystems: preprocessing, model training and testing, and model predicting as shown in FIG. 6.

The preprocessing subsystem of the present invention provides a mechanism to ingest and transform structured data, such as internal surveys, and unstructured data, such as external publicly-available reviews, for consumption.

In a preferred embodiment, the preprocessing subsystem has the ability to retrieve external data of the provider from a third party website or database. External data refers to data that is produced and/or hosted outside of the platform. Because of this, the external data is not structured in a manner that is readily ingestible by the preprocessing system and therefore may also be referred to as “unstructured” data herein.

The external data may be retrieved by requests made to a third party API or by scraping website data, preferably through standard Python libraries. In one embodiment, the external data of the provider is a publicly-available review of the provider authored by a reviewer that is a previous or current patient of the provider outside of the platform. The external data of the provider may also include state licensure certifications, infractions, the CMS performance metrics, Healthcare Effectiveness Data and Information Set (HEDIS) performance metrics, health plan claims data, along with detailed records relating to physician prescribing patterns, diagnoses codes, procedure codes, number of visits or consults, consult lengths, patient panel size, and other details from electronic health records. The present invention may retrieve and ingest any one of the external data noted above or a combination thereof. Each source of external data may be given its own score for a particular physician, or all sources of external data may be normalized and aggregated to provide a single score for the external data for a particular physician. Any set or subset of the external data may further be broken down and aggregated by one or more demographic data values of the reviewer or source of the external data.

In a preferred embodiment, the external data, particularly in the case of a publicly-available review, is parsed through a natural language processor (NLP) to probabilistically obtain a numeric sentiment value. In embodiments of the present invention, a sentiment value may be obtained by analyzing the external data through, for example, Python's NLTK library, and scored based on sentiment, for example, positive (pos.), negative (neg.), or neutral (neu.) as seen in Example 4. The overall process for NLP according to an embodiment of the present invention is outlined in FIG. 9. In embodiments of the present invention, a probabilistic determination of sentiment is deemed conclusive if the probability of accuracy is greater than 50%, 75%, 80%, 90%, 95%, or 99%.

In an embodiment of the present invention, the retrieved external data may then be stored in an “external review record” in a database, i.e., non-volatile memory of a computer system. The external review record may contain data such as the source of the external data, the retrieval time, and a provider identifier. Such an identifier may be a unique government-issued identifier, such as an NPI number. In a preferred embodiment, the external review record is correlated to the identifier of the provider to which it relates. In the case of publicly-available reviews of the provider, the external review record may also contain the text of the review, a numerical sentiment score value of the review, and one or more demographic data values of the reviewer, such as an age or age range of the reviewer, a gender of the reviewer, and a geographical location of the reviewer.

In a preferred embodiment, the preprocessing system also ingests internal data about the provider. Internal data refers to data and metadata generated by the patients/members and providers that are existing users of the platform rather than third party data. Because the creation of the internal data is controlled under the platform, it can be structured in a manner that is readily ingestible by the preprocessing subsystem, and is therefore may also be referred to as “structured” data herein.

It is preferred that the internal data is generated from structured surveys completed by historical patients of the provider. Suitable survey types include, but are not limited to, member satisfaction surveys, provider rating surveys, quality surveys, experience surveys, and the like. In another embodiment, the internal data may be metadata about the provider such as waiting times, length of consultations, and quality metrics. Metadata may be particularly available in a telehealth or internet-based medical care platform, where patient and provider encounters occur within a controlled environment between networked computers. Each source of internal data may be given its own score for a particular physician, or all sources of internal data may be normalized and aggregated to provide a single score for the internal data for a particular physician. Any set or subset of the internal data may further be broken down and aggregated by one or more demographic data values of the historical patient.

In an embodiment of the present invention, the internal data may be stored in an “internal review record” in a database, i.e., non-volatile memory of a computer system. The internal review record may contain data such as the source of the internal data, the completion time, and a provider identifier. Such an identifier may be a unique government-issued identifier, such as an NPI number. In a preferred embodiment, the internal review record is correlated to the identifier of the provider to which it relates. In the case of surveys completed by historical patients of the provider, the internal review record may also contain one or more ratings of the provider derived from the survey and one or more demographic data values of the historical patient, such as an age or age range of the historical patient, a gender of the historical patient, and a geographical location of the historical patient.

In an embodiment, the preprocessing system builds an index representation of the data by first ingesting the internal data and the external data of the provider. In an embodiment, the preprocessing system computes the index by taking one of two approaches depending on the originating data source, for example, structured surveys as the internal data and unstructured online reviews as the external data.

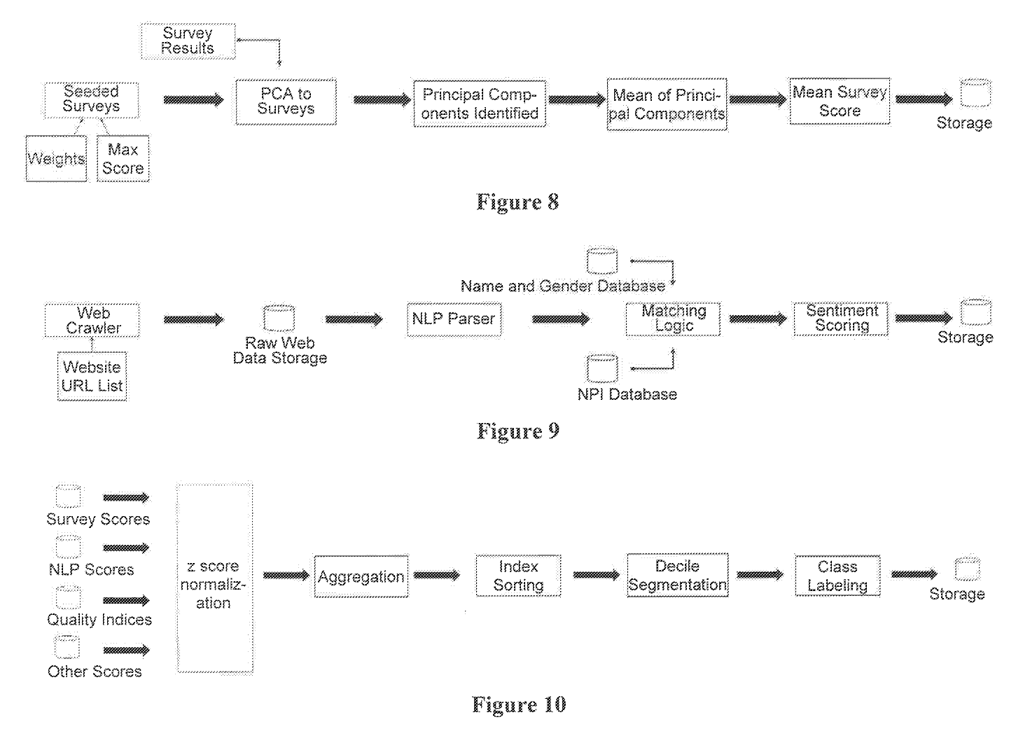

In one embodiment of the present invention, with respect to internal data in the form of structured surveys, the preprocessing subsystem is first seeded with the survey questions and responses, which are assigned a numerically weighted rating. The numerical weight may be adjusted as needed according to the particular requirements of the recommender system. Surveys completed with responses by past patients of the provider are ingested and the responses for each survey undergo principal component analysis (PCA) to generate a new set of variables as linear combinations of the original survey questions. This process explains the maximum amount of variation within the survey. The identified principle components, specifically the questions identified in the first principle component, are used to re-calculate a new variable summarizing the behavior in the original survey by taking a simple arithmetical average of the identified variables. The result is an average score for each survey type. This process is repeated for each survey type, generating a common scoring algorithm for every completed survey ingested by the system. Example 2 provides an example of the type of survey questions the system ingests and weights. The ingestion process for surveys is outlined in FIG. 8.

In one embodiment of the invention, with respect to external data in the form of unstructured publicly-available reviews, the preprocessing subsystem employs scraping to retrieve provider reviews by patients on a third-party provider review website or database such as Yelp, 1800dentist, and Healthgrades. In a preferred embodiment, the scraping relies on standardized Python libraries BeautifulSoup, Requests, and Natural Language Toolkit (NLTK). The scraped data is then ingested, stored and indexed within a persistent storage database, i.e., non-volatile memory of a computer system. In an embodiment, the text of the scraped reviews are parsed and data such as names, locations, and additional information about the review useful for sentiment analysis is tagged. In one embodiment, the provider names and/or patient names of the review are tagged for gender. If no gender is provided in the review, gender may be inferred by cross-referencing a database of names and genders, for example the genderize.io database, to probabilistically determine the patient's gender. In another embodiment, the provider names and/or patient names of the review are tagged for age. If no age is provided in the review, age may be inferred by cross-referencing a database of names and age ranges to probabilistically determine the patient's age range. An example of age ranges that may be used for categorization is provided in Example 3. In general, the name of the reviewer is analyzed to probabilistically determine age or gender by comparing the name to a large database of individual's names that have known associated genders and dates of birth. Such a database can be compiled, for example, through census records, birth records, and the like. In embodiments of the present invention, a probabilistic determination of age or gender is deemed conclusive if the probability of accuracy is greater than about 50%, 75%, 80%, 90%, 95%, or 99%.

In embodiments of the present invention, before the review is inserted into the database, matching of the review is conducted to ensure that the provider being reviewed is properly matched to the correct provider record in the recommender system. In such embodiments, data such as one or more of the reviewed provider's first name, last name, middle initial, middle name, gender, age, and geographic location may be compared to provider records in the database, or alternatively, one or more of the data may be used to query a third-party database, such as the NPI database, to retrieve the unique government-issued identifier of the provider, which is then used to determine a match among the provider records of the database.

In an embodiment of the present invention, the output of the preprocessing subsystem is an intermediary persistent table, or multidimensional feature space, as shown in Example 5. The feature space relates to one patient and defines one or more patient-provider combinations with each “row” or “tuple” representing a unique combination of that patient to a different provider. In accordance with the present invention, each patient-provider combination of the feature space may be defined by a set of values, i.e., independent variables, that may be selected from any of the fields of the patient record and/or provider record. In a preferred embodiment the set of values includes at least one demographic data value of the patient. In another embodiment, the set of values may also include at least one demographic data value of the provider. In addition, the set of values of a patient-provider combination may include the patient's prior rating(s) of the particular provider, if any. In an embodiment, the set of values may include a unique identifier to identify the provider of the patient-provider combination, such as a unique government-issued identifier. The set of values in an embodiment may also include a unique identifier to identify the patient.

In a particularly preferred embodiment, the set of values of the patient-provider combination includes one or more aggregate external review scores of the provider and/or one or more aggregate internal review scores of the provider. The aggregate internal review scores for the provider are derived from the internal review records corresponding to the provider, and similarly, the aggregate external review scores of the provider are derived from the external review records corresponding to the provider. In a preferred embodiment of the invention, only those internal review records associated with a historical patient having at least one same demographic data value as the patient of the feature space are used in computing the aggregate internal review score, and similarly, only those external review records associated with a reviewer having at least one same demographic data value with the patient of the feature space are used in computing the aggregate external review score. Thus, for example, if the patient being assessed in the feature space is male, only those internal review records associated with historical patients that are also male would be used in computing an aggregate internal review score. It is envisioned that multiple aggregate internal review scores and multiple aggregate external review scores will be incorporated in the feature space, each one aggregated according to a different demographic data value(s) of the patient.

In some cases, particularly where the provider is new to the platform, there will be no internal review records relating to the provider. In such cases a “cold start” problem is avoided by relying on the external review records of the provider. Thus, in an embodiment of the present invention, after a provider record is created for the new provider, external data of the provider is retrieved and external review records for the provider are created based on the retrieved external data.

In accordance with the goal of the present invention to maintain patient data separately to ensure confidentiality, preferably each generated feature space defines patient-provider combinations involving only a single patient. Thus, a generated feature space for a single patient preferably includes a combination of that patient with multiple different providers. In one embodiment, the feature space defines a patient-provider combination for one patient with every provider registered with the platform. In another embodiment, the feature space defines a patient-provider combination for one patient with all providers that share a common geographic location with the patient. That is, the provider is licensed to practice medicine in the same geographic location in which the patient resides or is located. Accordingly, in embodiments of the present invention, before the feature space is generated, a set of available providers is determined by filtering the total list of providers to those that share a common geographical location with the requesting patient, or are within close proximity, such as within about 5 miles, 10 miles, 20 miles, 50 miles, etc. In a further embodiment, the set of available providers can further be filtered to those that are currently available at the time of the patient request to conduct a consultation.

The purpose of modeling this patient-provider combination relationship is to predict the match probability of the i^{th} patient and j^{th} provider in a utility matrix, as shown in Example 1, with n patients and p providers. In exemplary embodiments of the present invention, the utility matrix is generated by evaluating historical medical outcomes, quality metrics, and member satisfaction surveys and reviews, for example.

In an exemplary embodiment, the composite scores of surveys, reviews, HEDIS measures, and other medical quality indices, for example, and other measures binding an optimal relationship are normalized through a basic z score formula, indicated as: z=(x−μ)/ρ, and aggregated by provider, patient age, gender, and date/time, for example, to develop a calculated index. The indices are sorted, for each patient-provider, from highest to lowest scores, divided into deciles, and assigned a numerical value, such as 1 for the top decile, 2 for the second decile, 3 for the third decile, and 4 for the remaining data. The problem then takes the form of a four-class classification problem, where we predict index categories based on a feature space developed through data harmonization. An example of the general categories for this feature space are represented in Example 6 and the transformation is outlined in FIG. 10.

In embodiments of the present invention the predictive engine classifies the patient-provider combinations into one of two or more classes, preferably three or more classes, and most preferably four or more classes. In an embodiment, it is preferred that the predictive engine classifies the patient-provider combinations into one of two, three, four, or five classes. Classification into two classes is referred to as binary classification while classification into three or more classes is referred to as multiclass classification. An example of a binary classification system according to the present invention could be a “good match” or a “bad match,” with a “good match” assigned a numerical value of 1 and a “bad match” assigned a numerical value of 0. An example of a multiclass classification system according to the present invention could be an “excellent match,” a “great match,” a “good match,” or a “bad match,” with an “excellent match” assigned a numerical value of 4, a “great match” assigned a numerical value of 3, a “good match” assigned a numerical value of 2, and a “bad match” assigned a numerical value of 1. Any other numerical values may be used as long as they are capable of differentiating between the classes. In embodiments of the present invention, it is preferred that one of the classifications contain optimal patient-provider matches. Using the examples above, the “good match” class would contain optimal matches in the binary example and the “excellent match” class would contain optimal matches in the multiclass example.

In embodiments of the present invention, a requesting patient is presented with an array of providers classified as optimal matches and preferably having the same geographic location as the patient, or within a close proximity thereof If no providers are classified in the class of optimal matches, the patient may be presented with an array of providers from the second-most optimal match classification, and so forth. Alternatively, a message may be transmitted to the patient indicating that no matching providers are currently available.

In an embodiment of the present invention, the model training and testing subsystem consumes, preferably asynchronously, the preprocessed data discussed above, commonly referred to as training data, as directed by the application service. The trained and verified model is then serialized and available for real-time model fitting.

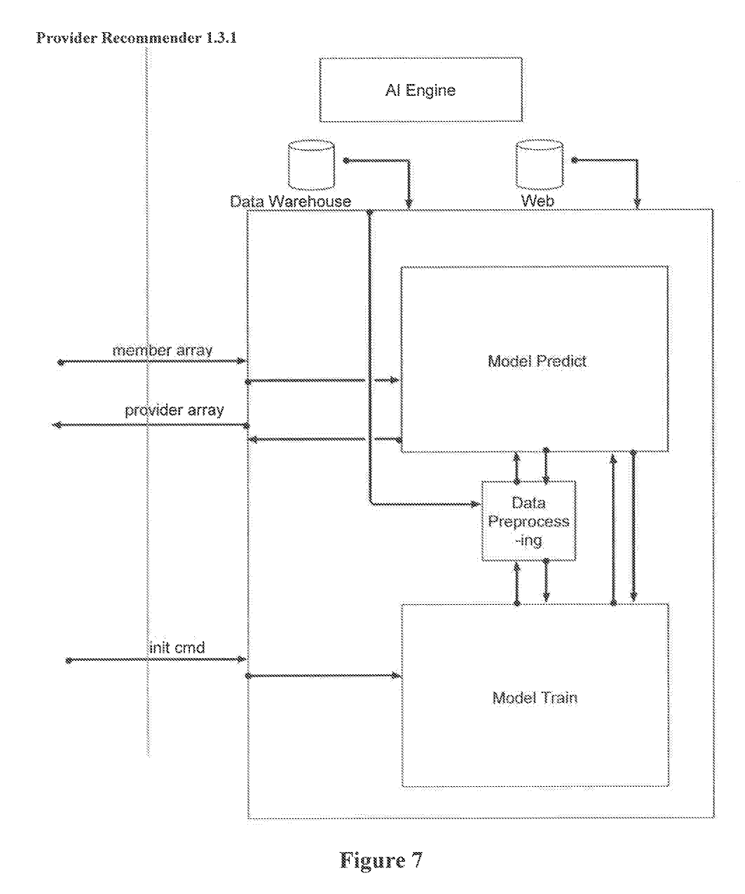

In an embodiment of the present invention, the model predicting subsystem scores the optimal provider match(es) for a specific patient/member. In a preferred embodiment, a request for a provider match is made through an application service request of the model predicting subsystem for a specific member/patient. The model fitting process also relies on preprocessing as seen in FIG. 7. Model predicting is the process of predicting an outcome by referencing properly formatted and preprocessed historical results, which have been previously evaluated through several statistical processing techniques and calculations, collectively referred to as machine learning, a subdomain of AI.

Various approaches of machine learning for a predictive engine are known to persons skilled in the art, such as decision tree learning, association rule learning, neural networks, inductive logic programming, support vector machines (SVM), clustering, Bayesian networks, reinforcement learning, representation learning, similarity and metric learning, sparse dictionary learning, rule-based machine learning, learning classifier systems, and feature selection approach. Any of the aforementioned approaches may be used, alone or in combination, with the predictive engine/AI engine of the present invention and would be within the level of ordinary skill in the art to implement.

In an embodiment, the predictive engine relies on the values of the feature space for determining optimal matches. Examples of the considerations of the predictive engine that should be specifically mentioned include whether the patient's and provider's gender match, whether the provider has historically seen patients having one or more of the patient's medical conditions, whether the provider has a high aggregate internal review score from historical patients that have the same medical condition(s)/gender/age range/race/ethnicity as the patient, whether the provider has a high aggregate external review score from reviewers that have the same medical condition(s)/gender/age range/race/ethnicity as the patient, and whether the patient has previously rated the provider highly.

In preferred embodiments of the present invention, the recommender system may be made portable and adaptable. By leveraging the concept of containers, the system of the present invention can be abstracted through the concept of bundling the application and deploying anywhere on any cloud platform or service provider.

Although the present invention is described with reference to matching patients with healthcare providers, it would be understood by a person skilled in the art that the systems and methods of the present invention can be adapted to match consumers with providers of other services, such as plumbers, electricians, carpenters, beauticians, consultants, lawyers, veterinarians, financial advisors, and the like.

The present invention also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, and magnetic disks, read-only memories (ROMs), random access memories (RAMs), EPROMs, EEPROMs, magnetic or optical cards, flash memories including USB keys with non-volatile memory or any type of media suitable for storing electronic instructions, each coupled to a computer system bus.

The present invention can take the form of an entirely hardware embodiment, an entirely software embodiment or an embodiment containing both hardware and software elements. In a preferred embodiment, the present invention is implemented in software, which includes but is not limited to firmware, resident software, microcode, etc.

Furthermore, the present invention can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening contact or public networks. Modems, cable modem and Ethernet cards are just a few of the currently available types of network adapters.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description above. In addition, the present invention is not described with reference to any particular programming language. It will be appreciated that a variety of programming languages may be used to implement the teachings of the specification as described herein.

Implementation of the invention may utilize software or graphical user interface designs other than that depicted in the Figures, as would be apparent to a person skilled in the art.

It is envisioned that any feature or element that is positively identified in this description may also be specifically excluded as a feature or element of an embodiment of the present invention as defined in the claims.

The invention described herein may be practiced in the absence of any element or elements, limitation or limitations which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the claims.

### EXAMPLES

### Example 1

The following table is an example of the patient-provider utility matrix that contains the transformed dependent variable used for classification of matches.

TABLE I
Patient-Provider Utility Matrix

| | Providerⱼ | Providerⱼ | Providerⱼ |
|---|---|---|---|
| Patientᵢ | 1 | 2 | 3 |
| Patientᵢ | 3 | 1 | 4 |
| Patientᵢ | 2 | 4 | 2 |

### Example 2

The following table is an example representation of the type of survey completed by a patient that may be used to compute an internal review score for a provider.

TABLE II
Patient Provider Survey Example

| | | Numerical |
|---|---|---|
| Question | Response | Weight |
| Did your consultation resolve your | Yes | 1 |
| immediate problem? | No | 0 |
| How would you rate the service | Outstanding | 2 |
| overall? | Good | 1 |
| | Poor | 0 |
| How likely are you to recommend to a | 10 | 10 |
| friend (Where 10 = Extremely Likely | 9 | 9 |
| and 1 = Not Likely At All)? | 8 | 8 |
| | . . . | . . . |
| Did the provider listen and understand | Outstanding | 2 |
| your problem? | Good | 1 |
| | Poor | 0 |
| Did you feel comfortable asking the | Yes | 2 |
| physician questions? | Somewhat | 1 |
| | Not at All | 0 |

### Example 3

The following table is an example of an age range breakdown suitable for use with the present invention.

TABLE III
Patient Age Ranges

| Age Logic | Age Range |
|---|---|
| Age <=17 | 0 to 17 |
| Age >17 and Age <=26 | 18 to 26 |
| Age >26 and Age <=30 | 27 to 30 |
| Age >30 and Age <=45 | 31 to 45 |
| Age >45 and Age <=55 | 46 to 55 |
| Age >55 and Age <=65 | 56 to 65 |
| Age >65 | 66+ |

### Example 4

The following table is an example of numerical sentiment scoring of example external provider reviews by natural language processing, which would be used to compute an external review score for a provider.

TABLE IV
Sentiment Score Example

| Parsed Review Text | Sentiment Score |
|---|---|
| I was first met by a gentleman who took my | neg: 0.34, neu: 0.61, |
| vitals then Dr. Smith saw me promptly | pos: 0.05 |
| afterwards. He only spent a minute with me | |
| and I felt like he was distracted during the | |
| exam. | |
| I will definitely return to Dr. Smith and for all | neg: 0.00, neu: 0.06, |
| regular check ups going forward as well. She | pos: 0.94 |
| was fantastic. | |

### Example 5

The following table is an abbreviated example of an intermediary persistent table with scored metrics stratified by age, gender, and time, and by a provider NPI.

TABLE V
Result Stratification Example

| | | | | | | Mean Pos. | Mean |
|---|---|---|---|---|---|---|---|
| Provider | | | Provider | Patient | Patient | Sentiment | Survey |
| NPI | Patient ID | Date/Time | Gender | Gender | Age | Score | Score |
| Providerⱼ | Patientᵢ | 00:00 00:00 | F | M | 27 to 30 | .88 | .75 |
| Providerⱼ | Patientᵢ | 00:00 00:00 | M | M | 46 to 55 | .75 | .55 |
| Providerⱼ | Patientᵢ | 00:00 00:00 | F | F | 18 to 26 | .35 | .85 |
| Providerⱼ | Patientᵢ | 00:00 00:00 | F | F | 31 to 45 | .60 | .30 |

### Example 6

The following table is an example of categories of factors that may be converted to features for predicting an optimal patient-provider match.

TABLE VI
Patient Provider Data Summary

| Category | Description |
|---|---|
| Patient | Age and gender |
| | Prior consult history |
| | Prior survey scores and ratings |
| Provider | Age and gender |
| | Specialties |
| | Prior survey scores and ratings |
| | Prior consult history |
| | Prior consult performance |
| Shared | Demographics and location |

### Example 7

The following table is an example of an abbreviated set of features transformed from raw visit data and surveys. The surveys measure the experience of providers and patients during visits within a virtual clinic setting.

TABLE VII
Example of Features

| Row | Name | Description | Equation |
|---|---|---|---|
| 1 | state_score_std | standard deviation of survey scores by state | $θ = \sqrt{\frac{1}{N - 1} {\sum }_{i = 1}^{N} {( {x}_{i} - \overline{x} )}^{2}}$ |
| 2 | state_gender_score_std | standard deviation of survey scores by state and gender | $θ = \sqrt{\frac{1}{N - 1} {\sum }_{i = 1}^{N} {( {x}_{i} - \overline{x} )}^{2}}$ |
| 3 | state_score_mean | mean of survey scores by state | $μ = {\sum }_{i = 1}^{N}$ |
| 4 | state_gender_score_mean | mean of survey scores by state and gender | $μ = {\sum }_{i = 1}^{N}$ |
| 5 | state_gender_age_score_mean | mean of survey scores by state, gender, age | $μ = {\sum }_{i = 1}^{N}$ |

1. A healthcare recommender system for matching healthcare providers with patients comprising:
one or more provider records, each provider record corresponding to a provider and comprising at least a unique government-issued identifier of the provider;
one or more patient records, each patient record corresponding to a patient and comprising at least one demographic data value of the patient;
a multidimensional feature space relating to one patient and defining one or more patient-provider combinations, wherein each patient-provider combination comprises a set of values that includes at least the demographic data value of the patient, and at least one of an aggregate internal review score of the provider and an aggregate external review score of the provider,
wherein the aggregate internal review score of the provider is derived from one or more internal review records corresponding to the provider, each internal review record including an internal score provided by a historical patient of the provider and a demographic data value of the historical patient, and the aggregate internal review score of the provider is computed from the internal review records having a demographic data value of the historical patient that is the same as the demographic data value of the patient,
wherein the aggregate external review score of the provider is derived from one or more external review records corresponding to the provider, each external review record including an external score provided by a reviewer and a demographic data value of the reviewer, and the aggregate external review score of the provider is computed from the external review records having a demographic data value of the reviewer that is the same as the demographic data value of the patient;
and a predictive engine capable of machine learning that classifies each patient-provider combination of the feature space into one of two or more classes according to the set of values of the patient-provider combination.

2. The healthcare recommender system of claim 1 wherein the demographic data value of the patient, historical patient, and reviewer is selected from the group consisting of gender, race, ethnicity, geographical location, age range, and medical condition.

3. The healthcare recommender system of claim 1 wherein the unique government-issued identifier of the provider is a National Provider Identifier issued by the Centers for Medicare and Medicaid Services.

4. The healthcare recommender system of claim 1 wherein the aggregate internal review score is computed from at least one structured survey in which the provider is assessed by the historical patient.

5. The healthcare recommender system of claim 1 wherein the aggregate external review score is a sentiment value computed from a natural language processing of at least one review of the provider authored by the reviewer and from a third party website or database.

6. The healthcare recommender system of claim 5 wherein a name of the reviewer is analyzed to determine an age and/or gender of the reviewer, and wherein at least one of the age and gender is the demographic data value of the reviewer.

7. A computer-implemented method for matching healthcare providers with patients comprising the steps:
(i) creating a provider record corresponding to a provider and populating the provider record with at least a unique government-issued identifier of the provider;
(ii) creating a patient record corresponding to a patient and populating the patient record with at least one demographic data value of the patient;
(iii) receiving one or more ratings of the provider from one or more historical patients of the provider, and for each rating creating an internal review record populated with the rating and a demographic data value of the historical patient, wherein the internal review record is correlated to the unique government-issued identifier of the provider;
(iv) retrieving one or more reviews of the provider authored by one or more reviewers from one or more third party websites or databases, and for each review: computing a numerical sentiment value by natural language processing of the review and determining at least one demographic data value of the reviewer, and creating an external review record populated with the sentiment value and the demographic data value of the reviewer, wherein the external review record is correlated to the unique government-issued identifier of the provider;
(v) generating a multidimensional feature space relating to one patient and defining one or more patient-provider combinations, wherein each patient-provider combination comprises a set of values that at least includes the demographic data value of the patient, an aggregate internal review score computed from the internal review records having a demographic data value of the historical patient that is the same as the demographic data value of the patient, and an aggregate external review score computed from the external review records having a demographic data value of the reviewer that is the same as the demographic data value of the patient; and
(vi) classifying by a predictive engine capable of machine learning each patient-provider combination of the feature space into one of two or more classes according to the set of values of the patient-provider combination.

8. The method of claim 7 wherein the demographic data value of the patient, historical patient, and reviewer is selected from the group consisting of gender, race, ethnicity, geographical location, age range, and medical condition.

9. The method of claim 7 wherein the unique government-issued identifier of the provider is a National Provider Identifier issued by the Centers for Medicare and Medicaid Services.

10. A computer-implemented method for matching a patient with one or more healthcare providers comprising the steps:
(i) receiving a request from the patient to be matched with the providers, wherein the patient has a corresponding patient record comprising at least one demographic data value;
(ii) generating a multidimensional feature space relating to the patient and defining one or more patient-provider combinations, wherein each patient-provider combination comprises a set of values that at least includes the demographic data value of the patient and at least one of an aggregate internal review score of the provider and an aggregate external review score of the provider,
wherein the aggregate internal review score of the provider is derived from one or more internal review records corresponding to the provider, each internal review record including an internal score provided by a historical patient of the provider and a demographic data value of the historical patient, and the aggregate internal review score of the provider is computed from the internal review records having a demographic data value of the historical patient that is the same as the demographic data value of the patient,
wherein the aggregate external review score of the provider is derived from one or more external review records corresponding to the provider, each external review record including an external score provided by a reviewer and a demographic data value of the reviewer, and the aggregate external review score of the provider is computed from the external review records having a demographic data value of the reviewer that is the same as the demographic data value of the patient;
(iii) classifying by a predictive engine capable of machine learning each patient-provider combination of the feature space into one of two or more classes according to the set of values of the patient-provider combination, wherein at least one class represents optimal provider matches;
(iv) presenting to the patient one or more providers classified in the class representing optimal provider matches; and
(v) receiving a selection of one provider from the patient.

11. The method of claim 10 wherein the demographic data value of the patient, historical patient, and reviewer is selected from the group consisting of gender, race, ethnicity, geographical location, age range, and medical condition.

12. The method of claim 10 wherein the unique government-issued identifier of the provider is a National Provider Identifier issued by the Centers for Medicare and Medicaid Services.

13. The method of claim 10 wherein the aggregate internal review score is computed from at least one structured survey in which the provider is assessed by the historical patient.

14. The method of claim 10 wherein the aggregate external review score is a sentiment value computed from a natural language processing of at least one review of the provider authored by the reviewer and from a third party website or database.

15. The method of claim 14 wherein a name of the reviewer is analyzed to determine an age and/or gender of the reviewer, and wherein at least one of the age and gender is the demographic data value of the reviewer.
